(19) United States
(12) Patent Application Publication
(10) Pub. No.: US 2021/0174911 A1
(43) Pub. Date: Jun. 10, 2021

(54) BLOCKCHAIN-BASED METHODS AND SYSTEMS

(71) Applicant: Scintillating, Inc., Acton, MA (US)

(72) Inventors: Kunal Sharda, Acton, MA (US); Kevin Liu, Boxborough, MA (US); William Robert Conrad Hemond, Acton, MA (US)

(21) Appl. No.: 17/112,973

(22) Filed: Dec. 4, 2020

Related U.S. Application Data
(63) Continuation of application No. PCT/US19/35466, filed on Jun. 4, 2019.
(60) Provisional application No. 62/680,187, filed on Jun. 4, 2018.

(51) Int. Cl. G16H 10/20; G06F 16/27; G16H 10/60; G06F 21/60; G06Q 20/40; H04L 9/00; G06F 16/182; G06F 21/62; G06Q 20/38

(52) U.S. Cl. CPC G16H 10/20; G06F 16/27; G16H 10/60; G06F 21/602; G06Q 2220/00; H04L 9/008; G06F 16/182; G06F 21/6254; G06Q 20/3829; G06Q 20/401

(57) ABSTRACT

A blockchain-based clinical study management system simplifies data regulation while enabling data transparency. The system identifies data falsification and guarantees monetary compensation for trial participants. The system records and manages informed consent histories for trial participants. Related methods, apparatuses, devices, techniques and articles are also described.

The present application is a continuation-in-part of International Application No. PCT/US2019/035466 filed Jun. 4, 2019, which claims priority from U.S. Provisional Application No. 62/680,187 filed Jun. 4, 2018, all of which applications are incorporated herein by reference in its entirety.

### BACKGROUND

Data generated from health research is stored on a centralized server, which can be secured both physically and cryptographically. Unfortunately, this creates issues with data regulation and transparency: researchers are burdened with the paperwork involving consent, data submission, and regulatory compliance. Thus, publicly releasing data publicly is impractical. For the participant, this creates undue stress, as they too must manage submitted forms and keep themselves informed about the study, while often receiving little personal communication from the researcher. On the other hand, the researcher has to handle the cost of the study's upkeep.

Also, in developed systems, consent is managed in the context of a single trial, where a patient enrolled in a trial gives their consent for their data to be collected for the context of that trial. This single trial method allows for single-party consent to be collected, but not retrieved and reused with compliant continuity.

Further, in developed systems, blockchain software systems require the user to manually acquire cryptocurrency and store it in a blockchain wallet.

The present inventors developed improvements in blockchain-based methods and systems that overcome at least the above-referenced problems with the methods and systems of the related art.

### SUMMARY

Improved blockchain-based methods and systems can provide a decentralized research study management system that tracks end-to-end data provenance and proves that collected data has not been tampered with. Leveraging blockchain immutability, it can provide a public, replicated audit log for data uploads, individual patient consents, and study administrative actions (such as inviting new researchers to collaborate). It would provide a secure and efficient way for scientists to share and build off of existing research datasets.

With a decentralized system there is an increased control over data and availability of payment regarding patient-based studies.

Methods for storing and retrieving data associated with a subject, may suitably comprise: a) receiving data and a verified consent from a subject; b) storing the data in a blockchain based distributed ledger; and c) retrieving the data from the blockchain-based distributed ledger. The verified consent can for instance provide consent to later use of the data by another party that may or may not be identified at the time of the consent being provided.

In exemplary embodiments, the data relates to the health or physical condition of the subject. For instance, the data may be retrieved for use in a clinical study of multiple subjects. The scientific study may be a clinical trial, for example a new treatment trial including a new therapeutic agent trial or study.

In an exemplary embodiment, notification is transmitted to the subject or user upon retrieving the subject's data from the blockchain based distributed ledger. For example, notification can be transmitted to the subject or user at the initiation of retrieving of the subject's data from the blockchain.

In particularly exemplary embodiments, the hash of the received data is deployed or stored on an Ethereum blockchain.

In certain aspects, the blockchain based distributed ledger is a private blockchain. In other aspects, the blockchain based distributed ledger is a public blockchain.

In additional exemplary embodiments, confirmation will be generated and received for example by an administrator of the blockchain that the representation of the data has been permanently added to the blockchain based distributed ledger.

In various aspects, the data from the subject is encrypted and hashed (cryptographic hash) and then that cryptographic hash is deployed on the blockchain based distributed ledger.

In certain embodiments, data stored on the blockchain based distributed ledger is under the control of a sponsor or administrator of a clinical trial. In other embodiments, data is stored in decentralized file storage system that suitably may not be under the control of a clinical trial sponsor. Suitable decentralized file storage system may optionally have computing nodes at the sponsor site or elsewhere.

In additional exemplary embodiments, storing the data in the blockchain based distributed ledger includes forming a transaction for transferring a quantity of currency to an address corresponding to the value.

In another embodiment, a blockchain interface system is provided that comprises a 1) processor; and 2) a computer readable medium storing machine-readable instructions that when executed by the processor to: i) receive data for a blockchain object where the data has been associated with a consent of a party associated with the data (in some embodiments, both data and verified consent is obtained before deploying or storing the data); and ii) deploy the data to a first blockchain. Suitably, the instructions further include to encrypt and hash (cryptographic hash) the received data.

In a yet further embodiment, software for storing scientific data associated with a scientific study (e.g. clinical trial) is provided, the software is stored in a non-transitory form on a computer-readable medium and including instructions for causing a computing system to: i) receive clinical data from a user where the data has been associated with a consent of a party associated with the data (in some embodiments, both data and consent particularly verified consent is obtained before storing the data); and ii) store the data in a blockchain based distributed ledger.

In a yet further embodiment, a blockchain-based software platform is provided that comprises: a. An openly-accessible database for participants to record medical and demographic information, and b. Smart contracts that allow for the creation and management of scientific studies, with data recorded on chain, and c. Smart contracts that allow for provable data auditing and traceability under an encrypted system, and d. A verification system allowing for data or medical record provenance to be validated using the blockchain.

In some exemplary embodiments, systems and methods may suitably comprise use of decentralized, blockchain-based smart contracts to: (1) create an open, filterable database listing participants interested in studies; (2) create a standardized method of medical data verification through attestation from medical professionals; (3) provide secure, immutable, and optionally encrypted data storage for medical studies; (4) automatically track and manage the consent process for all patients in a study without the use of paperwork; and/or (5) remove counterparty risk for participants by requiring full payment for patients to be paid upfront to the contract upon study creation.

In an aspect, we provide methods and systems that can allow an easier way for researchers to create these studies, and maintain them, including with government-mandated compliance.

In an exemplary embodiment, the platform also provides a standard, token-based method of data verification by medical professionals enabling study creators to verify their statistics.

Data immutability increases trust in research studies, which is particularly useful in patient based studies where data can be verified. If data is initially falsified, it can be traced to the inputter, and if it is later falsified, it will be recorded, thus discouraging falsification.

In another aspect, methods and systems are provided that can aid researchers and/or participants to create and enter studies on an efficient and convenient basis, while providing data transparency and standardized formats for transferability between studies.

In some exemplary embodiments, the systems provide meta-consent management and consent data aggregation.

In some exemplary embodiments, the systems provide privacy-preserving statistical analysis including for the purposes of regulatory compliance.

In some exemplary embodiments, the systems provide decentralized role-based access control (RBAC) including for purposes of granular clinical access permissions.

In some exemplary embodiments, the systems provide stablecoin-based trial payout in order to reduce counterparty risk.

A method for storing and retrieving data associated with a scientific study is provided. A device may be provided, the device may have at least one processor and a memory storing at least one program for execution by the at least one processor. The at least one program may include instructions, which, when executed by the at least one processor may cause the at least one processor to perform operations.

The operations may include receiving data with or without a verified consent from a subject. The operations may include storing the data in a blockchain based distributed ledger. The operations may include retrieving the data from the blockchain based distributed ledger.

Both data and a verified consent from a subject may be received.

Data may relate to the health or physical condition of the subject.

The data may be retrieved for use in a scientific study of multiple subjects.

A notification may be transmitted to the user upon initiating retrieving the subject's data from the blockchain based distributed ledger.

The hash of the received data may be deployed as a block chain object on an Ethereum blockchain.

The blockchain based distributed ledger may be a private blockchain.

The method may include receiving a confirmation that the block including the representation of the data has been permanently added to the blockchain based distributed ledger.

The received data may be encrypted and hashed to form a cryptographic hash and then deployed on the blockchain based distributed ledger.

The stored data may be under the control of a sponsor or administrator of the scientific study.

The stored data may be stored on a decentralized file storage system.

The decentralized data storage system may comprise one or more computing notes, which communicate to share and retrieve data.

The storing of the hash to form a cryptographic hash of the value on the blockchain based distributed ledger may include forming a transaction for transferring a quantity of currency.

A blockchain interface system is provided. The blockchain interface system may include a device having at least one processor and a memory storing at least one program for execution by the at least one processor. The at least one program may include instructions, when, executed by the at least one processor may cause the at least one processor to perform operations.

The operations may include receive data for a blockchain object with or without a consent of a party associated with the data.

The operations may include deploy the data to a first blockchain.

The instructions further include to encrypt and hash the received data to form a cryptographic hash.

A non-transitory computer-readable storage medium storing at least one program for storing scientific study data associated with a scientific study is provided. The at least one program may be for execution by at least one processor and a memory storing the at least one program. The at least one program may include instructions, when, executed by the at least one processor cause the at least one processor to perform operations.

The operations may include receiving scientific study data from a user where the data optionally has been associated with a consent of a party associated with the data.

The operations may include storing the data in a blockchain based distributed ledger.

A blockchain-based system is provided. The blockchain-based system may include at least one or more of the following: an openly-accessible database configured to store smart contracts; a verification system; and a device having at least one processor and a memory storing at least one program for execution by the at least one processor, the at least one program including instructions, when, executed by the at least one processor cause the at least one processor to perform operations.

The operations may include recording medical and demographic information in the openly-accessible database.

The operations may include recording the smart contracts for creation and management of scientific studies, with data recorded on chain.

The operations may include recording the smart contracts for provable data auditing and traceability under an encrypted system.

The operations may include validating with the verification system data or medical record provenance using the blockchain.

A blockchain-based system is provided. The blockchain-based system may include at least one or more of the following: a blockchain distributed network; a modular study system; a key management hierarchy system; a guaranteed transfer system; a consent management system; a decentralized architecture; an immutable and encrypted data storage system; a patient consent tracking and management system; a token-based reputation system; a distributed file storage system; a statistical analysis system; an encryption system; and a device having at least one processor and a memory storing at least one program for execution by the at least one processor, the at least one program including instructions, when, executed by the at least one processor cause the at least one processor to perform operations.

The operations may include storing and managing scientific studies using the blockchain distributed network.

The operations may include executing scientific studies using the modular study system and using the blockchain distributed network.

The operations may include delegating access permissions of scientific studies on chain using the key management hierarchy system.

The operations may include transferring of funds between users of the blockchain-based system without counterparty risk using the guaranteed transfer system.

The operations may include transferring of information between users of the blockchain-based system through explicit and/or opt-out consent using the consent management system.

The operations may include tracking the transfer of user information between participants of the blockchain-based system using the decentralized architecture.

The operations may include storing the scientific and medical studies accessible from the blockchain using the immutable and encrypted data storage system.

The operations may include tracking and managing patient consent using the patient consent tracking and management system by requiring cryptographic signatures from each user.

The operations may include validating submitted data and the quality of created studies using the token-based reputation system.

The operations may include contact addressing data access using the distributed file storage system by providing a hashed fingerprint of said data.

The operations may include statistically analyzing data stored on the distributed file system using the statistical analysis system.

The operations may include obscuring the value of each individual data point during analysis with homomorphic encryption techniques using the encryption system while preserving statistical validity.

A method for storing and retrieving data associated with a subject is provided. A device may be provided, the device may have at least one processor and a memory storing at least one program for execution by the at least one processor. The at least one program may include instructions, which, when executed by the at least one processor may cause the at least one processor to perform operations.

The operations may include storing clinical data from the subject in a secure smart contract.

The operations may include retrieving clinical data from the subject from the secure smart contract.

The data associated with the subject may relate to an informed consent collection of the subject.

The clinical data may be retrieved for use in clinical data management of multiple trials.

The clinical data may be retrieved for use in a multi-site clinical study.

The retrieved data may be deployed as a blockchain object on an Ethereum blockchain.

The blockchain object may be a blockchain based distributed ledger on a private blockchain.

A system is provided including a device having at least one processor and a memory storing at least one program for execution by the at least one processor, the at least one program including instructions, when, executed by the at least one processor cause the at least one processor to perform operations.

The operations may include receiving clinical data. The operations may include encrypting and hashing the received clinical data to form a cryptographic hash. The operations may include storing the data before deploying the cryptographic hash on a blockchain based distributed ledger.

The stored data may be used to track informed consent.

The stored data may be under control of a sponsor or administrator of a clinical trial, which generates clinical data.

Storing the cryptographic hash in the blockchain based distributed ledger may include forming a transaction for transferring a quantity of currency to an address corresponding to the cryptographic hash.

A blockchain interface system is provided. The blockchain interface system may include a device having at least one processor and a memory storing at least one program for execution by the at least one processor, the at least one program including instructions, when, executed by the at least one processor cause the at least one processor to perform operations.

The operations may include receiving data for a blockchain object where the data has been associated with a consent of a party associated with the data.

The operations may include deploying the data to a first blockchain.

The operations further include encrypting and hashing the received data to form a cryptographic hash.

A non-transitory computer-readable storage medium storing at least one program for storing clinical data associated with a clinical trial is provided, the at least one program for execution by at least one processor and a memory storing the at least one program, the at least one program including instructions, when, executed by the at least one processor cause the at least one processor to perform operations.

The operations may include receiving clinical data from a user where the data has been associated with a consent of a party associated with the data.

The operations may include storing the data in a blockchain based distributed ledger.

A blockchain-based system is provided. The blockchain-based system may include at least one or more of the following: an openly-accessible database configured to store smart contracts; a verification system; and a device having at least one processor and a memory storing at least one program for execution by the at least one processor, the at least one program including instructions, when, executed by the at least one processor cause the at least one processor to perform operations.

The operations may include recording clinical data and informed consent data to the openly accessible database.

The operations may include creating and managing clinical consent trails, with data recorded on chain using the smart contracts.

The operations may include provable data auditing and tracing under an encrypted system using the smart contracts.

The operations may include validating using the verification system trails of the informed consent data and the clinical data using the blockchain.

A blockchain-based system is provided. The blockchain-based system may include at least one or more of the following: a blockchain distributed network; a modular study system; a key management hierarchy system; a guaranteed transfer system; a consent management system; a decentralized architecture; an immutable and encrypted data storage system; a patient consent tracking and management system; a token-based reputation system; a distributed file storage system; a statistical analysis system; an encryption system; and a device having at least one processor and a memory storing at least one program for execution by the at least one processor, the at least one program including instructions, when, executed by the at least one processor cause the at least one processor to perform operations.

The operations may include storing and managing scientific studies using the blockchain distributed network.

The operations may include executing scientific studies using the modular study system and using the blockchain distributed network.

The operations may include delegating access permissions of scientific studies on chain using the key management hierarchy system.

The operations may include transferring of funds between users of the blockchain-based system without counterparty risk using the guaranteed transfer system.

The operations may include transferring of information between users of the blockchain-based system through explicit and/or opt-out consent using the consent management system.

The operations may include tracking the transfer of user information between participants of the blockchain-based system using the decentralized architecture.

The operations may include storing the scientific and medical studies accessible from the blockchain using the immutable and encrypted data storage system.

The operations may include tracking and managing patient consent using the patient consent tracking and management system by requiring cryptographic signatures from each user.

The operations may include validating submitted data and the quality of created studies using the token-based reputation system.

The operations may include contact addressing data access using the distributed file storage system by providing a hashed fingerprint of said data.

The operations may include statistically analyzing data stored on the distributed file system using the statistical analysis system.

The operations may include encrypting clinical data with end-to-end privacy-preserving encryption techniques using the encryption system to obscure the value of each individual data point during analysis while preserving statistical validity.

A non-transitory computer-readable storage medium storing at least one program for storing clinical study data associated with a clinical study is provided, the at least one program for execution by at least one processor and a memory storing the at least one program, the at least one program including instructions, when, executed by the at least one processor cause the at least one processor to perform operations.

The operations may include receiving clinical study data from a user with or without the data being associated with a consent of a party associated with the data.

The operations may include storing the data in a blockchain based distributed ledger by way of a smart contract.

A blockchain based system is provided. The blockchain based system may include at least one or more of the following: an openly-accessible database configured to store smart contracts; a verification system; and a device having at least one processor and a memory storing at least one program for execution by the at least one processor, the at least one program including instructions, when, executed by the at least one processor cause the at least one processor to perform operations.

The operations may include recording clinical information on the openly-accessible database.

The operations may include recording informed consent on the openly-accessible database.

The operations may include creating and managing clinical studies and trials, with data recorded on chain using the smart contracts.

The operations may include provable data auditing and tracing of the clinical studies and trials under an encrypted system using the smart contracts.

The operations may include validating using the verification system clinical data provenance and informed consent provenance using the blockchain.

The operations may include enabling and disabling operations based on predetermined needs of a specific clinical trial or site.

The operations may include storing user permissions for data and administrative actions of clinical trials on chain.

The operations may include guaranteed transferring of funds between users of the blockchain based system without counterparty risk.

The operations may include conditionally transferring information between uses of the blockchain based system based on explicit and/or opt-out consent management.

The operations may include tracking the transfer of user information between participants of the blockchain based system using decentralized architecture.

The operations may include storing immutable and encrypted data for the clinical trials accessible from the blockchain.

The operations may include tracking and managing patient consent by requiring cryptographic signatures from each user.

The operations may include validating submitted data and the quality of created studies using a token based reputation system.

The operations may include content-addressing data in a distributed file storage system. permitting data access by providing a hashed fingerprint of the addressed data.

The operations may include statistically analyzing data stored on the distributed file system.

The operations may include obscuring the value of each individual data point during analysis while preserving statistical validity using homomorphic encryption techniques.

The operations may include statistically analyzing data within a trusted execution environment and preserving the anonymity of individual data points.

The operations may include statistically analyzing data within a processor enclave and preserving the anonymity of individual data points.

The operations may include storing login credentials or other sensitive clinical data using an encryption method provided by a trusted execution environment.

The operations may include storing login credentials or other sensitive clinical data using an encryption method provided by a processor enclave and preserving the anonymity of individual data points.

The operations may include creating an inverted index or other search metadata to locate values in stored encrypted datasets and storing the inverted index or the other search metadata in an encrypted dataset.

The operations may include timestamping changes to study metadata using a public blockchain.

The operations may include proxying the connection between the blockchain and the web client using a backend server.

The operations may include Oasis Labs blockchain operations.

The operations may include sharing and reencrypting sensitive data between users of the platform using a blockchain.

The operations may include selecting varying levels of consent, including: consent only to collect data for a given study. The operations may include consent to share data about the given study with future researchers upon later authorization. The operations may include blanket consent to share data about the given study with future researchers without later authorization.

The operation may include revoking a participant's consent.

A system for clinical management is provided. The system may include a device having at least one processor and a memory storing at least one program for execution by the at least one processor, the at least one program including instructions, when, executed by the at least one processor cause the at least one processor to perform operations.

The operations may include providing blockchain-based informed meta-consent protocols.

The operations may include providing informed consent protocols for clinical trials, the informed consent protocols configured for trustable aggregate data usage and anonymization across multiple studies based on the blockchain-based informed meta-consent protocols,

The operations may include prompting a participant to select at least one of the informed consent protocols.

The informed consent protocols may include informed consent provenance, including operations including permitting researchers and auditors to review a history of a participant's consent on a decentralized network.

The informed consent protocols may include consent for the participant's data to be used with or without additional consent requests in follow-up studies, based on at least one or more of a field of study, a sponsor, or information relating to the study.

The informed consent protocols may include blanket consent for all of the participant's data to be used for any purpose, wherein the blanket consent selection may be stored on a decentralized network.

The informed consent protocols may include proactive consent for requests to be sent to the participant proactively using at least one or more of mobile push notifications, web notifications, or other electronic methods of notification and allowing for the consent requests to occur during or after a trial, decoupled from a trial duration.

A system for clinical management is provided. The system may include a device having at least one processor and a memory storing at least one program for execution by the at least one processor, the at least one program including instructions, when, executed by the at least one processor cause the at least one processor to perform operations.

The operations may include performing privacy-preserving statistical analysis requests on-chain or within a trusted execution environment on confidential clinical data, and without exposing raw contents of the confidential clinical data to any unencrypted storage system.

The operations may include preserving the anonymity of individual data points for regulatory compliance within clinical analysis.

A method of delegated access control within a clinical management system for achieving regulatory compliance is provided. A device may be provided, the device may have at least one processor and a memory storing at least one program for execution by the at least one processor. The at least one program may include instructions, which, when executed by the at least one processor may cause the at least one processor to perform operations.

The operations may include providing a plurality of hashed permissions for a respective plurality of levels of management within the clinical management system.

The operations may include uploading a plurality of individual data points associated with a clinical study to the memory.

The operations may include providing a plurality of data access permissions, each of the plurality of data access provisions corresponding to one of the plurality of the individual data points associated with the study.

The operations may include storing keyed encryption and metadata on a decentralized blockchain system.

A method of payment within a clinical trial management system to retain value is provided. A device may be provided, the device may have at least one processor and a memory storing at least one program for execution by the at least one processor. The at least one program may include instructions, which, when executed by the at least one processor may cause the at least one processor to perform operations.

The operations may include prompting and requiring a user to pay upfront using cryptocurrency to avoid counterparty risk.

The operations may include converting the paid cryptocurrency using blockchain smart contracts to and from the user's local currency without the user having to manually handle conversion steps.

The operations may include storing the paid cryptocurrency for a duration of the study as a stable cryptocurrency, which may be tied to a value of a US Dollar or other stable world currency.

Certain blockchain systems have been reported such as US2018/0218779, US2019/0013933, US 2016/0028552 and WO2018/057719, which are each fully incorporated herein by reference.

Other aspects of the improved blockchain-based methods and systems are discussed infra.

### BRIEF DESCRIPTION OF THE DRAWINGS

FIG. 1 is a general architecture diagram that shows the flow of payments and data for the lifecycle of a single study, according to an exemplary embodiment.

FIG. 2 shows how each step outlined by FIG. 1 maps onto smart contract changes that are persisted on the blockchain, according to an exemplary embodiment.

FIG. 3 diagrams the ways that a study can be concluded in a platform, by the action of for example either the creator or a supermajority of participants, according to an exemplary embodiment.

FIG. 4 further illustrates how researchers and users can interact in accordance with present systems and methods, according to an exemplary embodiment.

FIG. 5 is a schematic diagram of a computer device or system including at least one processor and a memory storing at least one program for execution by the at least one processor according to an exemplary embodiment.

It is noted that the drawings are not necessarily to scale. The drawings are intended to depict only typical aspects of the subject matter disclosed herein, and therefore should not be considered as limiting the scope of the disclosure. Those skilled in the art will understand that the structures, systems, devices, and methods specifically described herein and illustrated in the accompanying drawings are non-limiting exemplary embodiments and that the scope of the present invention is defined solely by the claims.

### DETAILED DESCRIPTION

The present systems and methods are suitably utilized within fields of health research analytics and data storage. The present methods and systems can be directed towards individuals who wish to create to create securable and scalable research studies, especially those related to the medical field, as current solutions are difficult to operate and offer little data transparency. Moreover, participants in such studies are often stymied by the hurdles necessary for them to submit personal health data, an issue the improved blockchain-based methods and systems aims to solve. Overall, the present methods and systems can aid both researchers and their participants in easily submitting and analyzing their data.

In some exemplary embodiments, systems and methods may include a key management hierarchy system that allows for tiered levels of data and administrative access control. Certain consent management also may be utilized where cryptographic consents are recorded directly on the blockchain.

In some exemplary embodiments, systems and methods also may include statistical analysis features, performed optionally with homomorphically-encrypted data to preserve privacy. Additionally, a token-based reputation system may be included such that participants in the systems or methods (e.g. participants or subjects that are providing data that is stored on the blockchain) can verify the authenticity and origin of created studies and researchers.

In systems and methods of some exemplary embodiments, first either (1) sensitive data fields of a data point is encrypted or (2) some or all data fields are encrypted using homomorphic encryption techniques.

In systems and methods of some exemplary embodiments, data mirroring in our system is done through the InterPlanetary File System where its authenticity can be validated, negating the need for a centralized clinical data silo.

In additional systems and methods of some exemplary embodiments, multi-factor verification can be utilized, including but not limited to verification through a decentralized token-based researcher and data reputation system, along with timestamp- and hash-based authentication.

In systems and methods of some exemplary embodiments, transactions or posted to a static address rather than a dynamically-generated recipient address

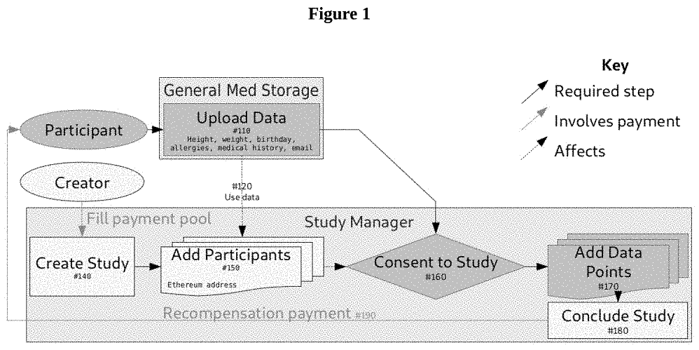

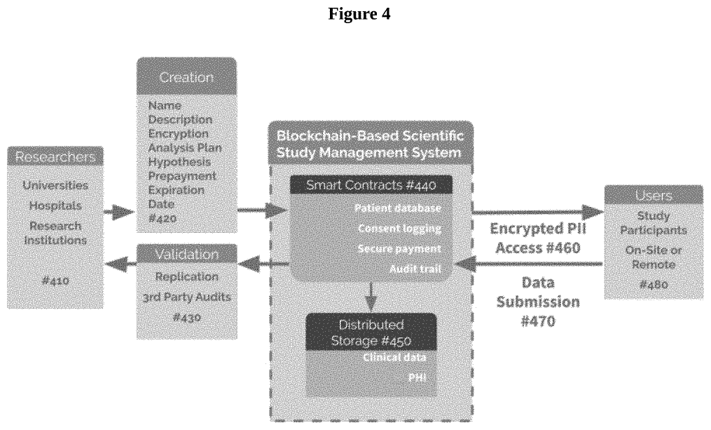

Referring now to the drawings, as shown generally in FIG. 1 and with regard to reference numbers shown in FIGS. 1 and 4, in an exemplary embodiment of the improved blockchain-based system, patients can provide various information such as basic demographic information to a smart contract 110, including but not limited to height, weight, age, and gender, agreeing to store it immutably on a blockchain-backed database in exchange for the ability to participate in studies created on the platform. For more casual studies, creators may then draw from this pool of participating users 120 to form research studies, putting in a pool of cryptocurrency capital 420 which will be paid out equally to all users when the study ends. If it is necessary for the study to be randomized, as in most professional ones, the creator may place certain parameters for entrance into their studies. Then, patients who fit this demographic as gleaned from their general information submitted, are automatically allowed to enter or apply to the study. Studies not involving participants may also be created on the platform, in which case the immutability and auditability guarantees are preserved for the study, but the participant database and consent mechanisms will not be used.

The study creator will also need to include important metadata about their research process, including but not limited to the name of the study, a detailed description of requirements, meeting times, or other parameters, a statistical analysis plan for the future data that is collected, a hypothesis or endpoints for the study, the expiration date, a citation, and a list of initial encryption public keys whose private keys are held by the study's researchers 420.

In the present systems, the creator will also have to submit a deposit equivalent to a fraction of the capital that they will pay out to participants if their study involves paid participants, which is held in the smart contract until study completion in order to ensure a continued, monetary interest in using the platform. They may then invite participants, and after cryptographically signing their consent to the study 160, a user may then be prompted to submit further specific data in order to satisfy the specific needs of the study.

Upon acceptance, these cryptographically signed participant consents are stored on the blockchain, which provides an immutable audit log to prove the validity of each consent. The consents may also be signed by the participant and stored on a remote server for later verification. Optionally, such consents may include meta-consent instructions for future data sharing, asking whether the user would be comfortable with sharing data collected in this study for future studies. User consent can also be revoked at any time by the user.

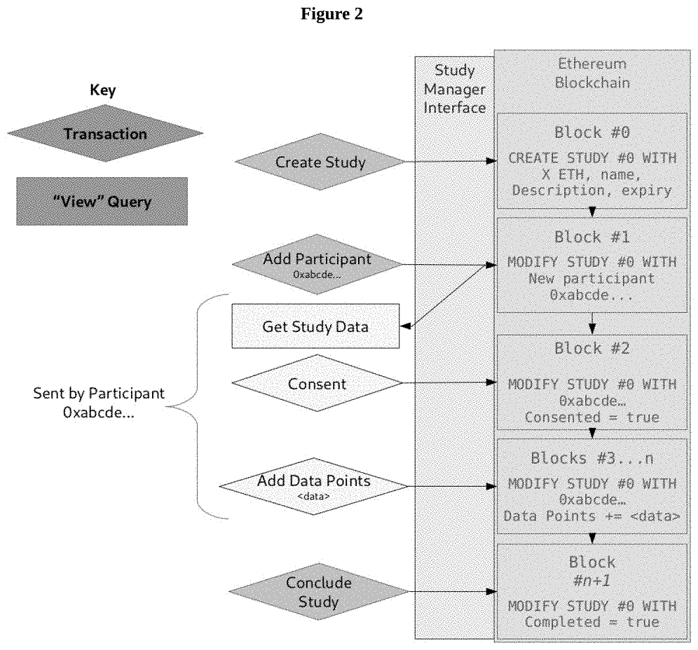

Then, after being accepted, either the researcher or the optional participants may submit data 440, 470. Data is encrypted using a public key algorithm including but not limited to elliptic-curve encryption techniques, from the client side to a list of encryption keys specified in the study metadata, which is updatable over the course of the study. Optionally, only specific values of each data point may be encrypted; certain others, such as those that do not include confidential or personally-identifiable information, may be included unencrypted to share the data publicly. Alternatively, the data may also be stored using homomorphic encryption, extending the encrypted data to allow for mathematical operations to be performed on the data without revealing the data itself. The partially- or wholly-encrypted data created by the encryption process may then be uploaded to a distributed file storage system, where data can be replicated across a network of independently-operated storage computers and addressed via the value of a one-way hash function on its contents. The hash produced as a result of this upload is then stored on blockchain smart contracts 170 (see also FIG. 2, Block #3). This encryption, upload, and hash storage process may be repeated as many times as necessary to upload an unbounded number of data points to the study.

Alternatively, the encrypted data may be uploaded to a blockchain network and distributed file system designed to store encrypted data and process it in secure enclaves (e.g. Intel SGX, Arm TrustZone, or AMD Secure Encrypted Virtualization), such as that of the company Oasis Labs. In this case, an address representing the location of the data on the blockchain is stored on a remote server, and this upload process may be repeated as many times as necessary to upload an unbounded number of data points to the study.

Upon data submission, automatic statistical analysis based on data stored on the distributed file system is provided to the researcher or any other authorized users (the authorization mechanism of which will be explained later), which includes, but is not limited to, graphs of the data, descriptive or bivariate statistics, or predictions for numerical outcomes. Such analysis may require the encryption key, if performed using conventional public-key encryption, or may be performed using the mathematical provisions of homomorphic encryption without decrypting the data itself.

Upon the eventual conclusion of the study 180, the funds given by the study creator are distributed equally to the users 190. This data flow is summarized in FIG. 1. Corresponding details of how data is represented on each block of the blockchain are summarized in FIG. 2.

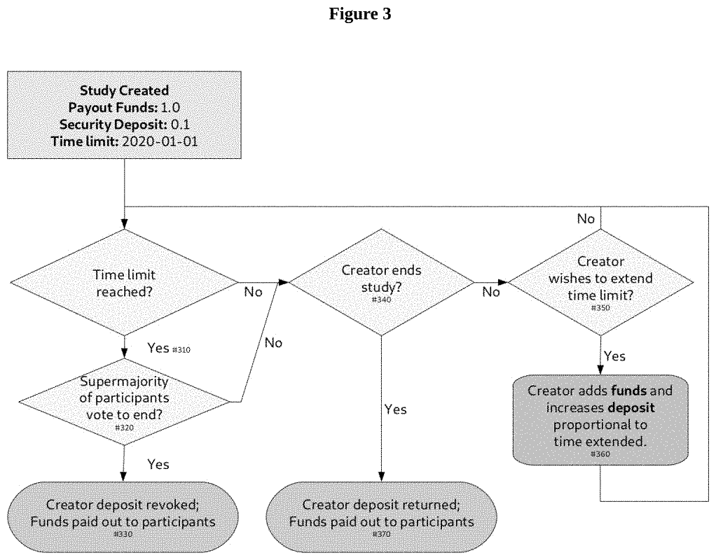

In the event that a study does not conclude 310, such as but not necessarily in an attempt to withhold payout, a vote by study participants can also end the study to force a payout #320, 330. This process is detailed in its majority in FIG. 3. As shown in FIGS. 1 and 3, study creators may choose to end the study at any time 180, 340, paying out participants and returning their deposit 190, 370. If the predetermined time limit of a study is reached without the creator ending the study, two-thirds of the participants may vote to end said study 320, at which point the study will end and pay out the payment pool to its users 330. The creator's deposit will then be confiscated and destroyed. However, to allow for outside factors that would cause a study to be delayed and thus legitimately require extra time, the study creator can also increase the amount of time in the study 350 by adding more capital and more deposit in a ratio equal to the ratio of funds initially given to time initially allocated 360. In order to compensate for this continued storage, a small fee is taken on this transaction, which may be either destroyed or used by the contract.

To discourage data falsification, both in the general medical data provided for the participant database and for more specific study data, data points may be verified by other users who hold a specific type of token. This token is distributed to trusted medical officials, such as doctors and study overseers, who are in direct contact with participants. These officials essentially stake their reputation tokens on the validity of the data points, thus incentivizing the submission of legitimate data. Study creators may filter by only verified entries in the participant database 120, or they may choose to pay out less in studies to participants who submit unverified data points.

During the study, researchers may also delegate permissions, including but not limited to those of data point submission, changing permissions of other users, adding participants, approving consented participants, or concluding the study. These permissions are also stored on the study management smart contract in an internal data structure specific to each study, or they may be stored on a remote server that uses blockchain timestamping technology to store a hash of the permissions on a public blockchain (e.g. Bitcoin, Ethereum).

In of some exemplary embodiments, the system allows for meta-consent management and consent data aggregation. Traditionally, consent is managed in the context of a single trial, where a patient enrolled in a trial gives their consent for their data to be collected for the context of that trial. This single trial method allows for single-party consent to be collected, but not retrieved and reused with compliant continuity. However, we use a blockchain-based protocol to allow patients to express meta-consent, namely the ability to control varying levels of data access by study type (e.g. for this study only, for other studies in this field, for all studies) as well as by organization (e.g. for this pharmaceutical sponsor only, for nonprofits only, for governments only). In a system of some exemplary embodiments, this granular consent is used to control data access for researchers who wish to aggregate data across multiple studies, ensuring that researchers are only able to technically access the data for which patients have properly consented to. Patients may also have the ability to be notified of data access requests through a secure, encrypted electronic notification mechanism, which in some embodiments may include mobile push notifications or desktop notifications. Upon acceptance, these cryptographically signed participant consents are stored on the blockchain, which provides an immutable audit log to prove the validity of each consent. The consents may also be signed by the participant and stored on a remote server for later verification. User consent can also be revoked at any time by the user. In of some exemplary embodiments, this allows for a blockchain-based informed consent system for the purpose of anonymized aggregate data usage across multiple studies.

In some exemplary embodiments, the system allows for privacy-preserving enclave statistical analysis for the purposes of regulatory compliance. Statistical analysis involves computations performed on a raw dataset that yield resultant data. Computing enclaves, also known as trusted execution engines (examples include, but are not limited to AMD Secure Encrypted Virtualization, Intel Software Guard Extensions, and Arm TrustZone), are specialized computer processors designed to run calculations without tampering or interference. We leverage computing enclaves to run statistical analysis on confidential clinical data whose raw contents are never exposed on any unencrypted storage system. In some exemplary embodiments, clinical data is transferred from the encrypted blockchain storage to the encrypted memory of the enclave, where the analysis request and the data are analyzed to see if the output dataset contains personally identifiable information. If the request and data pass validation, then data analysis is performed in the enclave. The output of the analysis is then stored again on the encrypted storage system. This analysis job may also be strictly access-controlled, such that only managers with valid analysis permissions for all of the datasets involved can perform the analysis. Once the analysis is performed, the resultant dataset (i.e. averages, graphs, or other analysis results) is no longer personally identifiable.

In some exemplary embodiments, the system allows for decentralized role-based access control (RBAC) for the purposes of granular clinical data access permissions. Role-based access control (RBAC) is an architecture of designing security systems such that users have access to perform certain actions or access data based on their role, which gives them a certain level of permissions. In exemplary embodiments, an RBAC system stored on-chain is used to control permissions for different levels of data access and management within a clinical system, so as to comply with regulatory requirements regarding data usage such as, but not limited to, the HIPAA Security Rule and the European Union General Data Protection Regulation (GDPR). In certain embodiments, these permissions are stored on the blockchain, such as but not included to a usage of smart contracts or hash keys. In some exemplary embodiments, these permissions are stored as metadata on the Ethereum blockchain associated with each data point uploaded, and for each permission change, the data is downloaded, re-encrypted with the new permission set, and reuploaded to the decentralized file storage network. In some exemplary embodiments, these permissions are stored as metadata associated with an encryption key that unlocks a dataset on the Oasis blockchain storage network, and access rights may be verified using untamperable software running in hardware enclaves. Changes to these permissions may be logged on-chain such that an auditor can later review changes to data access, such as but not limited to events, transaction logs, or other forms of recording historical actions. In particularly exemplary embodiments, this data control may be used to control data access on the level of individual data points uploaded to a study, with such control being exercised by a study administrator with appropriate permissions to manage data access.

In a fourth aspect, the system allows for stablecoin-based trial payout in order to reduce counterparty risk. Certain embodiments allow the transfer of cryptocurrency between users without devaluation over time. Traditionally, blockchain software systems require the user to manually acquire cryptocurrency and store it in a blockchain wallet. In some exemplary embodiments, however, the system may automatically, through the use of an iframe, redirect, or other software protocol, direct the user to purchase cryptocurrency using familiar currency, such as the US Dollar or other local currencies. In some exemplary embodiments, after the cryptocurrency has been purchased, the cryptocurrency is then converted automatically by smart contracts using a decentralized exchange (DEX) into a “stablecoin,” a coin whose value is tied directly to the US Dollar or other marker of stability, such as but not limited to the Dai Stablecoin System on the Ethereum blockchain. This conversion allows the cryptocurrency to hold value over time, with that value remaining predictable by researchers and participants. In some exemplary embodiments, the system also allows participants to withdraw this stablecoin, with smart contracts automatically converting the stablecoin with a DEX into the native currency of the underlying blockchain platform. This native currency may then be automatically sold for the user's local currency, again through the use of an iframe, redirect, or other software protocol. Certain embodiments thus use the above features to allow the creator of a study to seamlessly include a payment using a blockchain-based cryptocurrency to be given to participants upon study completion, for the purpose of eliminating counterparty risk by forcing the study creator to provide irreversible cryptocurrency payment at study creation, and for the participants of the study to seamlessly withdraw into their local currency.

FIG. 4 describes ways in which different users may interact with the blockchain-based scientific study management system. Researchers of many types, including those from universities, hospitals, and other research institutions 410 may create studies on the platform 420, including a variety of metadata. To do so, they interact through the means of a computer program, such as a web application, a mobile phone application, and/or a desktop application, which communicates through a computer network with the smart contract system 440 deployed on the blockchain distributed ledger. The contracts provide access to a database of potential participants, timestamped logs of the cryptographic consent transactions of participants, methods for secure payment using cryptocurrencies, and timestamped logs of each data point uploaded in a study, providing an audit trail 440. During a study's lifetime or after it has been marked as concluded, researchers can receive validation in a number of ways 430 on the data submitted to a study. For example, other researchers, given permission to access the raw data, may choose to replicate the study procedure, collect more data, and either confirm or contradict the study's findings. Furthermore, third-party auditors may also consult the raw data upon being given permission, where they can verify that users have properly given cryptographic consent on the blockchain-based study management system and that the data fits specified audit parameters.

Users interact with the platform in a different way depending on whether they are participating in an on-site or remote study 480. In the case of an on-site study, users will participate at the site of a research trial where the researcher(s) may input data points on behalf of the user 470. This data, potentially including personally identifiable information, is then partially or wholly encrypted based on the preference expressed by the researcher and stored in a persistent manner, including but not limited to storage on a distributed file storage system between a network of computing nodes 450. The hash (cryptographic hash) of this encrypted and stored data is then recorded using a blockchain transaction onto the smart contracts. The data is encrypted to both the researchers' and the user's keys, so that stored data can be decrypted either by the user or by researchers in the study 460. In the case of a remote study, users will submit their own data to a persistent storage system as described previously 450 after encryption, hashing, and recording it onto the blockchain as described previously 440.

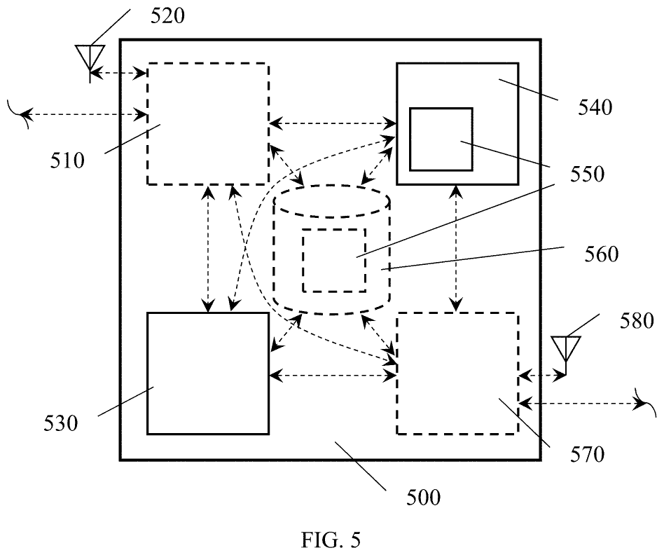

FIG. 5 is a schematic diagram of a computer device or system including at least one processor and a memory storing at least one program for execution by the at least one processor according to an exemplary embodiment. Specifically, FIG. 5 depicts a computer device or system 500 comprising at least one processor 530 and a memory 540 storing at least one program 550 for execution by the at least one processor 530. In some embodiments, the device or computer system 500 can further comprise a non-transitory computer-readable storage medium 560 storing the at least one program 550 for execution by the at least one processor 530 of the device or computer system 500. In some embodiments, the device or computer system 500 can further comprise at least one input device 510, which can be configured to send or receive information to or from any one of: an external device (not shown), the at least one processor 530, the memory 540, the non-transitory computer-readable storage medium 560, and at least one output device 570. The at least one input device 510 can be configured to wirelessly send or receive information to or from the external device via a means for wireless communication, such as an antenna 520, a transceiver (not shown) or the like. In some embodiments, the device or computer system 500 can further comprise at least one output device 570, which can be configured to send or receive information to or from any one from the group consisting of: an external device (not shown), the at least one input device 510, the at least one processor 530, the memory 540, and the non-transitory computer-readable storage medium 560. The at least one output device 570 can be configured to wirelessly send or receive information to or from the external device via a means for wireless communication, such as an antenna 580, a transceiver (not shown) or the like.

Each of the above identified modules or programs corresponds to a set of instructions for performing a function described above. These modules and programs (i.e., sets of instructions) need not be implemented as separate software programs, procedures or modules, and thus various subsets of these modules may be combined or otherwise re-arranged in various embodiments. In some embodiments, memory may store a subset of the modules and data structures identified above. Furthermore, memory may store additional modules and data structures not described above.

The illustrated aspects of the disclosure may also be practiced in distributed computing environments where certain tasks are performed by remote processing devices that are linked through a communications network. In a distributed computing environment, program modules can be located in both local and remote memory storage devices.

Moreover, it is to be appreciated that various components described herein can include electrical circuit(s) that can include components and circuitry elements of suitable value in order to implement the embodiments of the subject innovation(s). Furthermore, it can be appreciated that many of the various components can be implemented on at least one integrated circuit (IC) chip. For example, in one embodiment, a set of components can be implemented in a single IC chip. In other embodiments, at least one of respective components are fabricated or implemented on separate IC chips.

What has been described above includes examples of the embodiments of the present invention. It is, of course, not possible to describe every conceivable combination of components or methodologies for purposes of describing the claimed subject matter, but it is to be appreciated that many further combinations and permutations of the subject innovation are possible. Accordingly, the claimed subject matter is intended to embrace all such alterations, modifications, and variations that fall within the spirit and scope of the appended claims. Moreover, the above description of illustrated embodiments of the subject disclosure, including what is described in the Abstract, is not intended to be exhaustive or to limit the disclosed embodiments to the precise forms disclosed. While specific embodiments and examples are described herein for illustrative purposes, various modifications are possible that are considered within the scope of such embodiments and examples, as those skilled in the relevant art can recognize.

In particular and in regard to the various functions performed by the above described components, devices, circuits, systems and the like, the terms used to describe such components are intended to correspond, unless otherwise indicated, to any component which performs the specified function of the described component (e.g., a functional equivalent), even though not structurally equivalent to the disclosed structure, which performs the function in the herein illustrated exemplary aspects of the claimed subject matter. In this regard, it will also be recognized that the innovation includes a system as well as a computer-readable storage medium having computer-executable instructions for performing the acts and/or events of the various methods of the claimed subject matter.

The aforementioned systems/circuits/modules have been described with respect to interaction between several components/blocks. It can be appreciated that such systems/circuits and components/blocks can include those components or specified sub-components, some of the specified components or sub-components, and/or additional components, and according to various permutations and combinations of the foregoing. Sub-components can also be implemented as components communicatively coupled to other components rather than included within parent components (hierarchical). Additionally, it should be noted that at least one component may be combined into a single component providing aggregate functionality or divided into several separate sub-components, and any at least one middle layer, such as a management layer, may be provided to communicatively couple to such sub-components in order to provide integrated functionality. Any components described herein may also interact with at least one other component not specifically described herein but known by those of skill in the art.

In addition, while a particular feature of the subject innovation may have been disclosed with respect to only one of several implementations, such feature may be combined with at least one other feature of the other implementations as may be desired and advantageous for any given or particular application. Furthermore, to the extent that the terms “includes,” “including,” “has,” “contains,” variants thereof, and other similar words are used in either the detailed description or the claims, these terms are intended to be inclusive in a manner similar to the term “comprising” as an open transition word without precluding any additional or other elements.

As used in this application, the terms “component,” “module,” “system,” or the like are generally intended to refer to a computer-related entity, either hardware (e.g., a circuit), a combination of hardware and software, software, or an entity related to an operational machine with at least one specific functionality. For example, a component may be, but is not limited to being, a process running on a processor (e.g., digital signal processor), a processor, an object, an executable, a thread of execution, a program, and/or a computer. By way of illustration, both an application running on a controller and the controller can be a component. At least one component may reside within a process and/or thread of execution and a component may be localized on one computer and/or distributed between two or more computers. Further, a “device” can come in the form of specially designed hardware; generalized hardware made specialized by the execution of software thereon that enables the hardware to perform specific function; software stored on a computer-readable medium; or a combination thereof.

Moreover, the words “example” or “exemplary” are used herein to mean serving as an example, instance, or illustration. Any aspect or design described herein as “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

Computing devices typically include a variety of media, which can include computer-readable storage media and/or communications media, in which these two terms are used herein differently from one another as follows. Computer-readable storage media can be any available storage media that can be accessed by the computer, is typically of a non-transitory nature, and can include both volatile and nonvolatile media, removable and non-removable media. By way of example, and not limitation, computer-readable storage media can be implemented in connection with any method or technology for storage of information such as computer-readable instructions, program modules, structured data, or unstructured data. Computer-readable storage media can include, but are not limited to, RAM, ROM, EEPROM, flash memory or other memory technology, CD-ROM, digital versatile disk (DVD) or other optical disk storage, magnetic cassettes, magnetic tape, magnetic disk storage or other magnetic storage devices, or other tangible and/or non-transitory media which can be used to store desired information. Computer-readable storage media can be accessed by at least one local or remote computing device, e.g., via access requests, queries or other data retrieval protocols, for a variety of operations with respect to the information stored by the medium.

On the other hand, communications media typically embody computer-readable instructions, data structures, program modules or other structured or unstructured data in a data signal that can be transitory such as a modulated data signal, e.g., a carrier wave or other transport mechanism, and includes any information delivery or transport media. The term “modulated data signal” or signals refers to a signal that has at least one of its characteristics set or changed in such a manner as to encode information in at least one signal. By way of example, and not limitation, communication media include wired media, such as a wired network or direct-wired connection, and wireless media such as acoustic, RF, infrared and other wireless media.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Although at least one exemplary embodiment is described as using a plurality of units to perform the exemplary process, it is understood that the exemplary processes may also be performed by one or plurality of modules.

The use of the terms “first”, “second”, “third” and so on, herein, are provided to identify various structures, dimensions or operations, without describing any order, and the structures, dimensions or operations may be executed in a different order from the stated order unless a specific order is definitely specified in the context.

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for storing and retrieving data associated with a scientific study, wherein a device is provided, the device having at least one processor and a memory storing at least one program for execution by the at least one processor, the at least one program including instructions, which, when executed by the at least one processor cause the at least one processor to perform operations comprising:
receiving data with or without a verified consent from a subject;
storing the data in a blockchain based distributed ledger; and
retrieving the data from the blockchain based distributed ledger.

2. The method of claim 1 wherein both data and a verified consent from a subject are received.

3. The method of claim 1 wherein data relates to the health or physical condition of the subject.

4. The method of claim 1 wherein the data is retrieved for use in a scientific study of multiple subjects.

5. The method of claim 1 wherein a notification is transmitted to the user upon initiating retrieving the subject's data from the blockchain based distributed ledger.

6-17. (canceled)

18. A blockchain-based system comprising: a blockchain distributed network; and/or a modular study system; and/or a key management hierarchy system; and/or a guaranteed transfer system; and/or a consent management system; and/or a decentralized architecture; an immutable and encrypted data storage system; and/or a patient consent tracking and management system; and/or a token-based reputation system; and/or a distributed file storage system; and/or a statistical analysis system; and/or an encryption system; and a device having at least one processor and a memory storing at least one program for execution by the at least one processor, the at least one program including instructions, when, executed by the at least one processor cause the at least one processor to perform operations comprising:
storing and managing scientific studies using the blockchain distributed network, and/or executing scientific studies using the modular study system and using the blockchain distributed network, and/or delegating access permissions of scientific studies on chain using the key management hierarchy system, and/or
transferring of funds between users of the blockchain-based system without counterparty risk using the guaranteed transfer system, and/or
transferring of information between users of the blockchain-based system through explicit and/or opt-out consent using the consent management system, and/or
tracking the transfer of user information between participants of the blockchain-based system using the decentralized architecture, and/or
storing the scientific and medical studies accessible from the blockchain using the immutable and encrypted data storage system, and/or
tracking and managing patient consent using the patient consent tracking and management system by requiring cryptographic signatures from each user, and/or
validating submitted data and the quality of created studies using the token-based reputation system, and/or
contact addressing data access using the distributed file storage system by providing a hashed fingerprint of said data, and/or
statistically analyzing data stored on the distributed file system using the statistical analysis system, and/or
obscuring the value of each individual data point during analysis with homomorphic encryption techniques using the encryption system while preserving statistical validity.

19-32. (canceled)

33. A blockchain-based system comprising: a blockchain distributed network; and/or a modular study system; and/or a key management hierarchy system; and/or a guaranteed transfer system; and/or a consent management system; and/or a decentralized architecture; an immutable and encrypted data storage system; and/or a patient consent tracking and management system; and/or a token-based reputation system; and/or a distributed file storage system; and/or a statistical analysis system; and/or an encryption system; and a device having at least one processor and a memory storing at least one program for execution by the at least one processor, the at least one program including instructions, when, executed by the at least one processor cause the at least one processor to perform operations comprising:
storing and managing scientific studies using the blockchain distributed network, and/or executing scientific studies using the modular study system and using the blockchain distributed network, and/or
delegating access permissions of scientific studies on chain using the key management hierarchy system, and/or
transferring of funds between users of the blockchain-based system without counterparty risk using the guaranteed transfer system, and/or
transferring of information between users of the blockchain-based system through explicit and/or opt-out consent using the consent management system, and/or
tracking the transfer of user information between participants of the blockchain-based system using the decentralized architecture, and/or
storing the scientific and medical studies accessible from the blockchain using the immutable and encrypted data storage system, and/or
tracking and managing patient consent using the patient consent tracking and management system by requiring cryptographic signatures from each user, and/or
validating submitted data and the quality of created studies using the token-based reputation system, and/or
contact addressing data access using the distributed file storage system by providing a hashed fingerprint of said data, and/or
statistically analyzing data stored on the distributed file system using the statistical analysis system, and/or
encrypting clinical data with end-to-end privacy-preserving encryption techniques using the encryption system to obscure the value of each individual data point during analysis while preserving statistical validity.

34. (canceled)

35. A blockchain based system comprising: an openly-accessible database configured to store smart contracts; a verification system; and a device having at least one processor and a memory storing at least one program for execution by the at least one processor, the at least one program including instructions, when, executed by the at least one processor cause the at least one processor to perform operations comprising:
recording clinical information on the openly-accessible database,
recording informed consent on the openly-accessible database,
creating and managing clinical studies and trials, with data recorded on chain using the smart contracts,
provable data auditing and tracing of the clinical studies and trials under an encrypted system using the smart contracts, and
validating using the verification system clinical data provenance and informed consent provenance using the blockchain.

36. The blockchain based system of claim 35, the operations comprising: enabling and disabling operations based on predetermined needs of a specific clinical trial or site.

37. The blockchain based system of claim 35, the operations comprising: storing user permissions for data and administrative actions of clinical trials on chain.

38. The blockchain based system of claim 35, the operations comprising: guaranteed transferring of funds between users of the blockchain based system without counterparty risk.

39. The blockchain based system of claim 35, the operations comprising: conditionally transferring information between uses of the blockchain based system based on explicit and/or opt-out consent management.

40. The blockchain based system of claim 35, the operations comprising: tracking the transfer of user information between participants of the blockchain based system using decentralized architecture.

41. The blockchain based system of claim 35, the operations comprising: storing immutable and encrypted data for the clinical trials accessible from the blockchain.

42. The blockchain based system of claim 35, the operations comprising: tracking and managing patient consent by requiring cryptographic signatures from each user.

43. The blockchain based system of claim 35, the operations comprising: validating submitted data and the quality of created studies using a token based reputation system.

44. The blockchain based system of claim 35, the operations comprising: content-addressing data in a distributed file storage system; and permitting data access by providing a hashed fingerprint of the addressed data.

45. The blockchain based system of claim 35, the operations comprising: statistically analyzing data stored on the distributed file system.

46. The blockchain based system of claim 35, the operations comprising: obscuring the value of each individual data point during analysis while preserving statistical validity using homomorphic encryption techniques.

47. The blockchain based system of claim 35, the operations comprising: statistically analyzing data within a trusted execution environment and preserving the anonymity of individual data points.

48-61. (canceled)
